(19) United States
(12) Patent Application Publication
(10) Pub. No.: US 2002/0094954 A1
(43) Pub. Date: Jul. 18, 2002

(54) Human schizophrenia gene

(75) Inventors: Hreinn Stefansson, Krokamyri (IS); Valgerdur Steinthorsdottir, Silfurteigur (IS); Jeffrey R. Gulcher, Chicago, IL (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics, ehf,

(21) Appl. No.: 09/795,686

(22) Filed: Feb. 28, 2001

Related U.S. Application Data
(63) Continuation-in-part of application No. 09/515,715, filed on Feb. 28, 2000.

(51) Int. Cl. A61K 38/17; C12Q 1/68; C07H 21/04; C12N 9/00; C12P 21/02; C12N 5/06

(52) U.S. Cl. 514/12; 435/6; 435/183; 435/69.1; 435/325; 435/320.1; 536/23.2

(57) ABSTRACT

Nucleic acids comprising the neuregulin-1-associated gene 1 (NRG1AG1) and encoding NRG1AG1 polypeptides are disclosed. Also described are related nucleic acids encoding NRG1AG1 polypeptides; NRG1AG1 polypeptides; antibodies that bind to NRG1AG1 polypeptides; methods of diagnosis of susceptibility to schizophrenia; assays for agents that alter the activity of NRG1AG1 polypeptide or which identify NRG1AG1 binding agents, and the agents or binding agents identified by the assays; NRG1AG1 therapeutic agents, including the NRG1AG1 nucleic acids, NRG1AG1 polypeptides, or agents that alter the activity of an NRG1AG1 polypeptides; pharmaceutical compositions comprising the NRG1AG1 therapeutic agents; as well as methods of therapy of schizophrenia.

### RELATED APPLICATION

This application is a continuation-in-part of U.S. application Ser. No. 09/515,715, filed Feb. 28, 2000. The entire teachings of the above application is incorporated herein by reference.

### BACKGROUND OF THE INVENTION

Schizophrenia is a devastating form of psychopathology, with a lifetime prevalence worldwide of 0.5%-1%. Twin and adoption studies suggest that both genetic and environmental factors influence susceptibility (see, e.g., Tsuang, M. T. et aL, Schizophr. Res. 4(2):157-71 (1991); Tienari, P. J. and Wynne, L. C., Ann. Med. 26(4):233-7 (1994); Franzek, E. and Beckmann, H., Am. J. Psychiatry 155(1):76-83 (1998); Tsuang, M. T., J. Biomed. Sci. 5(1):28-30 (1998)). Among first-degree relatives, the risk has been reported to vary from 6% in parents, to 10% in siblings, and to 13% in children of schizophrenic individuals; if one of the parents is also schizophrenic, the risk to siblings increases to 17%, and children of two schizophrenics have a risk of 46% of developing the illness (McGue, M. and Gottesmann, I. I., Eur. Arch. Psychiatry Clin. Neurosci 240:174-181 (1991); see also, e.g., Lim, L. C. and Sim, L. P., Singapore Med. J. 33(6):645-7 (1992)). The mode of transmission, however, remains uncertain.

Reports of suggestive linkage to several loci have been published, including loci on chromosomes 3, 5, 6, 8, 10, 13, 20, 22 and the X chromosome (see, e.g., for chromosomes 3p and 8p, Pulver, A. E., et al., Am. J. Med. Genet. 60(4):252-60 (1995); for chromosomes 5q, 6p and 8p, Kendler, K. S. et al., Am. J. Med. Genet. 88(1):29-33 (1999); for chromosomes 5q, 6p, 8p, 20p and 22q, Hovatta, I. et al., Mol. Psychiatry 3(5):452-7 (1998); for chromosome 6p, Schwab, S. G. et al., Nat. Genet. 11(3):325-7 (1995), Brzustowicz, L. M. et al., Am. J. Hum. Genet. 61(6):1388-96 (1997) and Cao, Q. et al., Genomics 43(1):1-8 (1997); for chromosomes 6 and 8, Straub, R. E. et al., Cold Spring Harbor Symp. Quant. Biol 61I:823-33 (1996); for chromosome 8, Kendler, KS. et al., Am. J. Psychiatry 153(12):1534-40 (1996); for chromosome 10, Straub, R. E. et al., Am. J. Med. Genet. 81(4):296-301 (1998) and Schwab, S. G. et al., Am. J. Med. Genet. 81(4):302-307 (1998); for chromosome 13, Lin, M. W. et al., Psyciatr. Genet. 5(3):117-26 (1995); Lin, M. W. et al., Hum. Genet. 99(3):417-420 (1997) and Blouin, J. L. et al., Nat. Genet. 20(1):70-73 (1993) (8and 13); for chromosome 22, Gill, M. et al., Am. J. Med. Genet. 67(1):40-45 (1996) and Bassett, A. S. et al., Am. J. Med. Genet. 81(4):328-37 (1998); and for the X chromosome, Milunsky, J. et al., Clin. Genet. 55(6):455-60 (1999)).

### SUMMARY OF THE INVENTION

The present invention relates to isolated nucleic acid molecules comprising the neuregulin-1-associated gene 1 (NRG1AG1). In one embodiment, the isolated nucleic acid molecule comprises a nucleotide sequence selected from the group consisting of SEQ ID NO: 1 and the complement of SEQ ID NO: 1. The invention further relates to a nucleic acid molecule which hybridizes under high stringency conditions to a nucleotide sequence selected from the group consisting of SEQ ID NO: 1 and the complement of SEQ ID NO: 1. The invention additionally relates to isolated nucleic acid molecules (e.g., cDNA molecules) encoding an NRG1AG1 polypeptide (e.g., encoding SEQ ID NO: 6, 7, 8 or 9, or another splicing variant of NRG1AG1 polypeptide).

The invention further provides a method for assaying a sample for the presence of a nucleic acid molecule comprising all or a portion of NRG1AG1 in a sample, comprising contacting said sample with a second nucleic acid molecule comprising a nucleotide sequence encoding an NRG1AG1 polypeptide (e.g., SEQ ID NO: 1 or the complement of SEQ ID NO: 1; a nucleotide sequence encoding SEQ ID NO: 6, 7, 8, or 9, or another splicing variant of NRG1AG1 polypeptide), or a fragment or derivative thereof, under conditions appropriate for selective hybridization. The invention additionally provides a method for assaying a sample for the level of expression of an NRG1AG1 polypeptide, or fragment or derivative thereof, comprising detecting (directly or indirectly) the level of expression of the NRG1AG1 polypeptide, fragment or derivative thereof.

The invention also relates to a vector comprising an isolated nucleic acid molecule of the invention operatively linked to a regulatory sequence, as well as to a recombinant host cell comprising the vector. The invention also provides a method for preparing a polypeptide encoded by an isolated nucleic acid molecule described herein (an NRG1AG1 polypeptide), comprising culturing a recombinant host cell of the invention under conditions suitable for expression of said nucleic acid molecule.

The invention further provides an isolated polypeptide encoded by isolated nucleic acid molecules of the invention (e.g., NRG1AG1 polypeptide), as well as fragments or derivatives thereof. In a particular embodiment, the polypeptide comprises the amino acid sequence of SEQ ID NO: 6, SEQ ID NO: 7, SEQ ID NO: 8 or SEQ ID NO: 9. In another embodiment, the polypeptide is another splicing variant of an NRG1AG1 polypeptide. The invention also relates to an isolated polypeptide comprising an amino acid sequence which is greater than about 90 percent identical to the amino acid sequence of SEQ ID NO: 6, SEQ ID NO: 7, SEQ ID NO: 8 or SEQ ID NO: 9.

The invention also relates to an antibody, or an antigen-binding fragment thereof, which selectively binds to a polypeptide of the invention, as well as to a method for assaying the presence of a polypeptide encoded by an isolated nucleic acid molecule of the invention in a sample, comprising contacting said sample with an antibody which specifically binds to the encoded polypeptide.

The invention further relates to methods of diagnosing a predisposition to schizophrenia. The methods of diagnosing a predisposition to schizophrenia in an individual include detecting the presence of a mutation in NRG1AG1, as well as detecting alterations in expression of an NRG1AG1 polypeptide, such as the presence of different splicing variants of NRG1AG1 polypeptides. The alterations in expression can be quantitative, qualitative, or both quantitative and qualitative.

The invention additionally relates to an assay for identifying agents which alter (e.g., enhance or inhibit) the activity or expression of one or more NRG1AG1 polypeptides. For example, a cell, cellular fraction, or solution containing an NRG1AG1 polypeptide or a fragment or derivative thereof, can be contacted with an agent to be tested, and the level of NRG1AG1 polypeptide expression or activity can be assessed. The activity or expression of more than one NRG1AG1 polypeptides can be assessed concurrently (e.g., the cell, cellular fraction, or solution can contain more than one type of NRG1AG1 polypeptide, such as different splicing variants, and the levels of the different polypeptides or splicing variants can be assessed).

In another embodiment, the invention relates to assays to identify polypeptides which interact with one or more NRG1AG1 polypeptides. In a yeast two-hybrid system, for example, a first vector is used which includes a nucleic acid encoding a DNA binding domain and also an NRG1AG1 polypeptide, splicing variant, or fragment or derivative thereof, and a second vector is used which includes a nucleic acid encoding a transcription activation domain and also a nucleic acid encoding a polypeptide which potentially may interact with the NRG1AG1 polypeptide, splicing variant, or fragment or derivative thereof (e.g., a NRG1AG1 polypeptide binding agent or receptor). Incubation of yeast containing both the first vector and the second vector under appropriate conditions allows identification of polypeptides which interact with the NRG1AG1 polypeptide or fragment or derivative thereof, and thus can be agents which alter the activity of expression of an NRG1AG1 polypeptide.

Agents that enhance or inhibit NRG1AG1 polypeptide expression or activity are also included in the current invention, as are methods of altering (enhancing or inhibiting) NRG1AG1 polypeptide expression or activity by contacting a cell containing NRG1AG1 and/or polypeptide, or by contacting the NRG1AG1 polypeptide, with an agent that enhances or inhibits expression or activity of NRG1AG1 or polypeptide.

Additionally, the invention pertains to pharmaceutical compositions comprising the nucleic acids of the invention, the polypeptides of the invention, and/or the agents that alter activity of NRG1AG1 polypeptide. The invention further pertains to methods of treating schizophrenia, by administering NRG1AG1 therapeutic agents, such as nucleic acids of the invention, polypeptides of the invention, the agents that alter activity of NRG1AG1 polypeptide, or compositions comprising the nucleic acids, polypeptides, and/or the agents that alter activity of NRG1AG1 polypeptide.

### BRIEF DESCRIPTION OF THE DRAWINGS

FIG. 1 is a graphic representation of the nonparametric multipoint LOD score for the schizophrenia locus on 8p21-11.

FIG. 2 depicts haplotypes found in individuals affected with schizophrenia. Portions which are found in multiple haplotypes are depicted by backward slashes.

FIG. 3 depicts the order of sequenced BACS and boundaries for at-risk haplotypes for schizophrenia at locus 8p12.

FIG. 4 depicts the exons, single nucleotide polymorphisms (SNPs), and exons of neuregulin 1-associated gene 1 at locus 8p12. Cylinders, screened for mutations; N, new exons; open stars, SNPs (coding); filled stars, SNPs (untranslated); open circles, 5′ exons; filled circles, 3′ exons; lines, genomic neighbors.

### DETAILED DESCRIPTION OF THE INVENTION

As described herein, Applicants have used linkage and haplotype analyses to identify a disease susceptibility gene for schizophrenia residing in a 1.5 Mb segment on chromosome 8p12. The gene is neuregulin-1-associated gene 1 (NRG1AG1). The full sequence of the neuregulin 1-associated gene 1 is shown in Appendix I. Microsatellite markers and single nucleotide polymorphisms (SNPs) in the sequence are shown in Appendix II. Appendix III shows the splice variants for neuregulin 1-associated gene 1 exons.

### NUCLEIC ACIDS OF THE INVENTION

Accordingly, the invention pertains to an isolated nucleic acid molecule comprising the mammalian (e.g., primate or human) neuregulin-1-associated gene 1 (NRG1AG1). The term, “NRG1AG1,” as used herein, refers to an isolated nucleic acid molecule in the 8p21-11 locus, which is associated with a susceptibility to schizophrenia, and also to an isolated nucleic acid molecule (e.g., cDNA or the gene) that encodes an NRG1AG1 polypeptide (e.g., the polypeptide having SEQ ID NO: 6, 7, 8 or 9, as shown in Appendix I, or another splicing variant of an NRG1AG1 polypeptide). In a preferred embodiment, the isolated nucleic acid molecule comprises SEQ ID NO: 1 (shown in Appendix I) or the complement of SEQ ID NO: 1. In another preferred embodiment, the isolate nucleic acid molecule comprises the sequence of SEQ ID NO: 1 or the complement of SEQ ID NO: 1, except that one or more single nucleotide polymorphisms as shown in Appendix II are also present.

The isolated nucleic acid molecules of the present invention can be RNA, for example, mRNA, or DNA, such as cDNA and genomic DNA. A “neuregulin 1-associated gene 1 nucleic acid” (“NRG1AG1-nucleic acid”), as used herein, refers to a nucleic acid molecule (RNA, mRNA, cDNA, or genomic DNA, either single- or double-stranded) encoding NRG1AG1. DNA molecules can be double-stranded or single-stranded; single stranded RNA or DNA can be either the coding, or sense, strand or the non-coding, or antisense, strand. The nucleic acid molecule can include all or a portion of the coding sequence of the gene and can further comprise additional non-coding sequences such as introns and non-coding 3′ and 5′ sequences (including regulatory sequences, for example). Additionally, the nucleic acid molecule can be fused to a marker sequence, for example, a sequence that encodes a polypeptide to assist in isolation or purification of the polypeptide. Such sequences include, but are not limited to, those which encode a glutathione-S-transferase (GST) fusion protein and those which encode a hemagglutinin A (HA) polypeptide marker from influenza.

An “isolated” nucleic acid molecule, as used herein, is one that is separated from nucleic acids which normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules which are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence which is synthesized chemically or by recombinant means. Therefore, recombinant DNA contained in a vector are included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis

The present invention also pertains to variant nucleic acid molecules which are not necessarily found in nature but which encode an NRG1AG1 polypeptide (e.g., a polypeptide having the amino acid sequence of SEQ ID NO: 6, 7, 8, or 9, or another splicing variant of NRG1AG1 polypeptide). Thus, for example, DNA molecules which comprise a sequence that is different from the naturally-occurring nucleotide sequence but which, due to the degeneracy of the genetic code, encode an NRG1AG1 polypeptide of the present invention are also the subject of this invention. The invention also encompasses nucleotide sequences encoding portions (fragments), or encoding variant polypeptides such as analogues or derivatives of the NRG1AG1 polypeptide. Such variants can be naturally-occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions. Preferably the nucleotide (and/or resultant amino acid) changes are silent or conserved; that is, they do not alter the characteristics or activity of the NRG1AG1 polypeptide. In one preferred embodiment, the nucleotide sequences are fragments that comprise one or more polymorphic microsatellite markers (e.g., as shown in Appendix II). In another preferred embodiment, the nucleotide sequences are fragments that comprise one or more single nucleotide polymorphisms in the NRG1AG1 gene (e.g., as shown in Appendix II).

Other alterations of the nucleic acid molecules of the invention can include, for example, labelling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequences via hydrogen bonding and other chemical interactions. Such molecules include, for example, those in which peptide linkages substitute for phosphate linkages in the backbone of the molecule.

The invention also pertains to nucleic acid molecules which hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one embodiment, the invention includes variants described herein which hybridize under high stringency hybridization conditions (e.g., for selective hybridization) to a nucleotide sequence comprising a nucleotide sequence selected from SEQ ID NO: 1 or the complement of SEQ ID NO: 1. In another embodiment, the invention includes variants described herein which hybridize under high stringency hybridization conditions (e.g., for selective hybridization) to a nucleotide sequence encoding an amino acid sequence selected from SEQ ID NO: 6, 7, 8, or 9. In a preferred embodiment, the variant which hybridizes under high stringency hybridizations has an activity of NRG1AG1 (e.g., binding activity).

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2XSSC, 0.1XSSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology, 200:546-556 (1991). Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of ˜17° C. Using these guidelines, the washing temperature can be determined empirically for high, moderate or low stringency, depending on the level of mismatch sought.

For example, a low stringency wash can comprise washing in a solution containing 0.2XSSC/0.1% SDS for 10 min at room temperature; a moderate stringency wash can comprise washing in a prewarmed solution (42° C.) solution containing 0.2XSSC/0. 1% SDS for 15 min at 42° C.; and a high stringency wash can comprise washing in prewarmed (68° C.) solution containing O.1XSSC/0.1% SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, preferably at least 40%, more preferably at least 60%, and even more preferably at least 70%, 80% or 90% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the NBLAST and XBLAST programs (version 2.0) as described in Altschul et al., Nucleic Acids Res., 25:389-3402 (1997). When utilizing BLAST and Gapped BLAST programs, the default parameters of the respective programs (e.g., NBLAST) can be used. See http://www.ncbi.nlm.nih.gov. In one embodiment, parameters for sequence comparison can be set at score=100, wordlength=12, or can be varied (e.g., W=5 or W=20).

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) PNAS, 85:2444-8.

In another embodiment, the percent identity between two amino acid sequences can be accomplished using the GAP program in the CGC software package (available at http://www.cgc.com) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the CGC software package (available at http://www.cgc.com), using a gap weight of 50 and a length weight of 3.

The present invention also provides isolated nucleic acid molecules that contain a fragment or portion that hybridizes under highly stringent conditions to a nucleotide sequence comprising a nucleotide sequence selected from SEQ ID NO: 1 and the complement of SEQ ID NO: 1, and also provides isolated nucleic acid molecules that contain a fragment or portion that hybridizes under highly stringent conditions to a nucleotide sequence encoding an amino acid sequence selected from SEQ ID NO: 6, 7, 8, or 9. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, which encode antigenic polypeptides described herein are particularly useful, such as for the generation of antibodies as described below.

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science, 254, 1497-1500 (1991). As also used herein, the term “primer” in particular refers to a single-stranded oligonucleotide which acts as a point of initiation of template-directed DNA synthesis using well-known methods (e.g., PCR, LCR) including, but not limited to those described herein.

Typically, a probe or primer comprises a region of nucleotide sequence that hybridizes to at least about 15, typically about 20-25, and more typically about 40, 50 or 75, consecutive nucleotides of a nucleic acid molecule comprising a contiguous nucleotide sequence selected from: SEQ ID NO: 1, the complement of SEQ ID NO: 1, or a sequence encoding an amino acid sequence selected from SEQ ID NO: 6-9. In preferred embodiments, a probe or primer comprises 100 or fewer nucleotides, preferably from 6 to 50 nucleotides, preferably from 12 to 30 nucleotides. In other embodiments, the probe or primer is at least 70% identical to the contiguous nucleotide sequence or to the complement of the contiguous nucleotide sequence, preferably at least 80% identical, more preferably at least 90% identical, even more preferably at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

Representative oligonucleotides useful as probes or primers include the microsatellite markers shown in Appendix II.

The nucleic acid molecules of the invention such as those described above can be identified and isolated using standard molecular biology techniques and the sequence information provided in SEQ ID NO: 1, 6, 7, 8, and/or 9. For example, nucleic acid molecules can be amplified and isolated by the polymerase chain reaction using synthetic oligonucleotide primers designed based on one or more of the sequences provided in SEQ ID NO: 1 and/or the complement of SEQ ID NO: 1, or designed based on nucleotides based on sequences encoding one or more of the amino acid sequences provided in SEQ ID NO: 6, 7, 8, and/or 9. See generally PCR Technology: Principles and Applications for DNA Amplification (ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics, 4:560 (1989), Landegren et al., Science, 241:1077 (1988), transcription amplification (Kwoh et al., Proc. Natl. Acad. Sci. USA, 86:1173 (1989)), and self-sustained sequence replication (Guatelli et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be radiolabelled and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the polypeptide and the DNA encoding the polypeptide can be isolated, sequenced and further characterized.

Antisense nucleic acid molecules of the invention can be designed using the nucleotide sequences of SEQ ID NO: 1 and/or the complement of SEQ ID NO: 1, and/or a portion of SEQ ID NO: 1 or the complement of SEQ ID NO: 1, and/or a sequence encoding the amino acid sequence or SEQ ID NO: 6, 7, 8, and/or 9, or encoding a portion of SEQ ID NO: 6, 7, 8, and/or 9, and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

In general, the isolated nucleic acid sequences of the invention can be used as molecular weight markers on Southern gels, and as chromosome markers which are labeled to map related gene positions. The nucleic acid sequences can also be used to compare with endogenous DNA sequences in patients to identify genetic disorders (e.g., a predisposition for or susceptibility to schizophrenia), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Another aspect of the invention pertains to nucleic acid constructs containing a nucleic acid molecule selected from the group consisting of SEQ ID NO: 1 and the complement of SEQ ID NO: 1 (or a portion thereof). Yet another aspect of the invention pertains to nucleic acid constructs containing a nucleic acid molecule encoding the amino acid sequence of SEQ ID NO: 6, 7, 8, or 9. The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention (e.g, an exogenous NRG1AG1 gene, or an exogenous nucleic acid encoding an NRG1AG1 polypeptide) has been introduced. Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley (1991) Current Opinion in Bio/Technology, 2:823-829 and in PCT Publication Nos. WO 90/11354, WO 91/01140, WO 92/0968, and WO 93/04169. Clones of the non-human transgenic animals described herein can also be produced according to the methods described in Wilmut et al. (1997) Nature, 385:810-813 and PCT Publication Nos. WO 97/07668 and WO 97/07669.

### POLYPEPTIDES OF THE INVENTION

The present invention also pertains to isolated polypeptides encoded by NRG1AG1 (“NRG1AG1 polypeptides”), and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleotide sequence selected from the group consisting of SEQ ID NO: 1 and complements and portions thereof, e.g., SEQ ID NO: 6, SEQ ID NO: 7, SEQ ID NO: 8 or SEQ ID NO: 9, or a portion of SEQ ID NO: 6, 7, 8, or 9. However, the polypeptides of the invention also encompass fragments and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleotide sequence selected from the group consisting of SEQ ID NO: 1 and complements and portions thereof, or having substantial homology to a polypeptide encoded by a nucleic acid molecule comprising a nucleotide sequence selected from the group consisting of nucleotide sequences encoding SEQ ID NO: 6, 7, 8, and 9. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, typically at least about 70-75%, more typically at least about 80-85%, and most typically greater than about 90% or more homologous or identical. A substantially homologous amino acid sequence, according to the present invention, will be encoded by a nucleic acid molecule hybridizing to SEQ ID NO: 1, or portion thereof, under stringent conditions as more particularly described above, or will be encoded by a nucleic acid molecule hybridizing to a nucleic acid sequence encoding SEQ ID NO: 6, 7, 8, or 9, or portion thereof, under stringent conditions as more particularly described thereof.

To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity”. The percent homology between the two sequences is a function of the number of identical positions shared by the sequences (i.e., percent homology equals the number of identical positions/total number of positions times 100).

The invention also encompasses polypeptides having a lower degree of identity but having sufficient similarity so as to perform one or more of the same functions performed by a polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol., 224:899-904 (1992); de Vos et al. Science, 255:306-312 (1992)).

The invention also includes polypeptide fragments of the polypeptides of the invention. Fragments can be derived from a polypeptide encoded by a nucleic acid molecule comprising SEQ ID NO: 1 or a portion thereof and the complements thereof (e.g., SEQ ID NO: 6, 7, 8, or 9, or other splicing variants). However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example β-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al, The Journal of Biological Chemistry, 270,16:9459-9471 (1995). Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

In general, polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding agent, e.g., receptor or ligand, such as, for example, in an interaction trap assay, and to screen for peptide or small molecule antagonists or agonists of the binding interaction.

### ANTIBODIES OF THE INVENTION

In another aspect, the invention provides antibodies to the polypeptides and polypeptide fragments of the invention, e.g., having an amino acid sequence encoded by SEQ ID NO: 6, 7, 8, 9, or a portion thereof, or having an amino acid sequence encoded by a nucleic acid molecule comprising all or a portion of SEQ ID NO: 1 (e.g., SEQ ID NO: 6, 7, 8, 9, or another splicing variant, or portion thereof). The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lemer (1981) Yale J. Biol. Med., 54:387-402. Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J, 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

### DIAGNOSTIC AND SCREENING ASSAYS OF THE INVENTION

The present invention also pertains to diagnostic assays for assessing NRG1AG1 gene expression, or for assessing activity of NRG1AG1 polypeptides of the invention. In one embodiment, the assays are used in the context of a biological sample (e.g., blood, serum, cells, tissue) to thereby determine whether an individual is afflicted with schizophrenia, or is at risk for (has a predisposition for or a susceptibility to) developing schizophrenia. The invention also provides for prognostic (or predictive) assays for determining whether an individual is susceptible to developing schizophrenia. For example, mutations in the gene can be assayed in a biological sample. Such assays can be used for prognostic or predictive purpose to thereby prophylactically treat an individual prior to the onset of symptoms associated with schizophrenia. Another aspect of the invention pertains to assays for monitoring the influence of agents (e.g., drugs, compounds or other agents) on the gene expression or activity of polypeptides of the invention, as well as to assays for identifying agents which bind to NRG1AG1 polypeptides. These and other assays and agents are described in further detail in the following sections.

Diagnostic Assays

The nucleic acids, probes, primers, polypeptides and antibodies described herein can be used in methods of diagnosis of a susceptibility to schizophrenia, as well as in kits useful for diagnosis of a susceptibility to schizophrenia.

In one embodiment of the invention, diagnosis of a susceptibility to schizophrenia is made by detecting a polymorphism in NRG1AG1. The polymorphism can be a mutation in NRG1AG1, such as the insertion or deletion of a single nucleotide, or of more than one nucleotide, resulting in a frame shift mutation; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such mutation may be present in a single gene. Such sequence changes cause a mutation in the polypeptide encoded by NRG1AG1. For example, if the mutation is a frame shift mutation, the frame shift can result in a change in the encoded amino acids, and/or can result in the generation of a premature stop codon, causing generation of a truncated polypeptide. Alternatively, a polymorphism associated with a susceptibility to schizophrenia can be a synonymous mutation in one or more nucleotides (i.e., a mutation that does not result in a change in the polypeptide encoded by NRG1AG1). Such a polymorphism may alter splicing sites, affect the stability or transport of MRNA, or otherwise affect the transcription or translation of the gene. NRG1AG1 that has any of the mutations described above is referred to herein as a “mutant gene.”

In a first method of diagnosing a susceptibility to schizophrenia, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for, or carrying a defect for, schizophrenia (the “test individual”). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in NRG1AG1 is present, and/or to determine which splicing variant(s) encoded by NRG1AG1 is present. The presence of the polymorphism or splicing variant(s) can be indicated by hybridization of the gene in the genomic DNA, RNA, or cDNA to a nucleic acid probe. A “nucleic acid probe”, as used herein, can be a DNA probe or an RNA probe; the nucleic acid probe can contain at least one polymorphism in NRG1AG1 or contains a nucleic acid encoding a particular splicing variant of NRG1AG1. The probe can be any of the nucleic acid molecules described above (e.g., the gene, a fragment, a vector comprising the gene, a probe or primer, etc.)

To diagnose a susceptibility to schizophrenia, a hybridization sample is formed by contacting the test sample containing NRG1AG1, with at least one nucleic acid probe. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mNRA or genomic DNA. For example, the nucleic acid probe can be all or a portion of SEQ ID NO: 1, or the complement of SEQ ID NO: 1, or a portion thereof; or can be a nucleic acid encoding all or a portion of SEQ ID NO: 6, 7, 8, or 9. Other suitable probes for use in the diagnostic assays of the invention are described above (see, e.g., probes and primers discussed under the heading, “Nucleic Acids of the Invention”).

The hybridization sample is maintained under conditions which are sufficient to allow specific hybridization of the nucleic acid probe to NRG1AG1. “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and NRG1AG1 in the test sample, then NRG1AG1 has the polymorphism, or is the splicing variant, that is present in the nucleic acid probe. More than one nucleic acid probe can also be used concurrently in this method. Specific hybridization of any one of the nucleic acid probes is indicative of a polymorphism in NRG1AG1, or of the presence of a particular splicing variant encoded by NRG1AG1, and is therefore diagnostic for a susceptibility to schizophrenia.

In Northern analysis (see Current Protocols in Molecular Biology, Ausubel, F. et al., eds., John Wiley & Sons, supra), the hybridization methods described above are used to identify the presence of a polymorphism or of a particular splicing variant, associated with a susceptibility to schizophrenia. For Northern analysis, a test sample of RNA is obtained from the individual by appropriate means. Specific hybridization of a nucleic acid probe, as described above, to RNA from the individual is indicative of a polymorphism in NRG1AG1, or of the presence of a particular splicing variant encoded by NRG1AG1, and is therefore diagnostic for a susceptibility to schizophrenia.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed to specifically hybridize to a gene having a polymorphism associated with a susceptibility to schizophrenia. Hybridization of the PNA probe to NRG1AG1 is diagnostic for a susceptibility to schizophrenia.

In another method of the invention, mutation analysis by restriction digestion can be used to detect a mutant gene, or genes containing a polymorphism(s), if the mutation or polymorphism in the gene results in the creation or elimination of a restriction site. A test sample containing genomic DNA is obtained from the individual. Polymerase chain reaction (PCR) can be used to amplify NRG1AG1 (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the mutation or polymorphism in NRG1AG1, and therefore indicates the presence or absence of this susceptibility to schizophrenia.

Sequence analysis can also be used to detect specific polymorphisms in NRG1AG1. A test sample of DNA or RNA is obtained from the test individual. PCR or other appropriate methods can be used to amplify the gene, and/or its flanking sequences, if desired. The sequence of NRG1AG1, or a fragment of the gene, or cDNA, or fragment of the cDNA, or mRNA, or fragment of the mNRA, is determined, using standard methods. The sequence of the gene, gene fragment, cDNA, cDNA fragment, mNRA, or mNRA fragment is compared with the known nucleic acid sequence of the gene, cDNA (e.g., SEQ ID NO: 1, or a nucleic acid sequence encoding SEQ ID NO: 6, 7, 8, or 9, or a fragment thereof) or mRNA, as appropriate. The presence of a polymorphism in NRG1AG1 in37 dicates that the individual has a susceptibility to schizophrenia.

Allele-specific oligonucleotides can also be used to detect the presence of a polymorphism in NRG1AG1, through the use of dot-blot hybridization of amplified oligonucleotides with allele-specific oligonucleotide (ASO) probes (see, for example, Saiki, R. et al., (1986), Nature (London) 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to NRG1AG1, and that contains a polymorphism associated with a susceptibility to schizophrenia. An allele-specific oligonucleotide probe that is specific for particular polymorphisms in NRG1AG1 can be prepared, using standard methods (see Current Protocols in Molecular Biology, supra). To identify polymorphisms in the gene that are associated with a susceptibility to schizophrenia, a test sample of DNA is obtained from the individual. PCR can be used to amplify all or a fragment of NRG1AG1, and its flanking sequences. The DNA containing the amplified NRG1AG1 (or fragment of the gene) is dot-blotted, using standard methods (see Current Protocols in Molecular Biology, supra), and the blot is contacted with the oligonucleotide probe. The presence of specific hybridization of the probe to the amplified NRG1AG1 is then detected. Specific hybridization of an allele-specific oligonucleotide probe to DNA from the individual is indicative of a polymorphism in NRG1AG1, and is therefore indicative of a susceptibility to schizophrenia.

In another embodiment, arrays of oligonucleotide probes that are complementary to target nucleic acid sequence segments from an individual, can be used to identify polymorphisms in NRG1AG1. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips.™.,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT application Ser. No. WO 90/15070) and Fodor et al., PCT Publication No. WO 92/10092 and U.S. Pat. No. 5,424,186, the entire teachings of each of which are incorporated by reference herein. Techniques for the synthesis of these arrays using mechanical synthesis methods are described in, e.g., U.S. Pat. No. 5,384,261, the entire teachings of which are incorporated by reference herein.

Once an oligonucleotide array is prepared, a nucleic acid of interest is hybridized with the array and scanned for polymorphisms. Hybridization and scanning are generally carried out by methods described herein and also in, e.g., Published PCT application Ser. Nos. WO 92/10092 and WO 95/11995, and U.S. Pat. No. 5,424,186, the entire teachings of which are incorporated by reference herein. In brief, a target nucleic acid sequence which includes one or more previously identified polymorphic markers is amplified by well known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternate arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional description of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect polymorphisms in NRG1AG1 or splicing variants encoded by NRG1AG1. Representative methods include direct manual sequencing (Church and Gilbert, (1988), Proc. Natl. Acad. Sci. USA 81:1991-1995; Sanger, F. et al. (1977) Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al. (19891) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (CMC) (Cotton et al. (1985) Proc. Natl. Acad. Sci. USA 85:4397-4401); RNase protection assays (Myers, R. M. et al. (1985) Science 230:1242); use of polypeptides which recognize nucleotide mismatches, such as E. coli mutS protein; allele-specific PCR, for example.

In another embodiment of the invention, diagnosis of a susceptibility to schizophrenia can also be made by examining expression and/or composition of an NRG1AG1 polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by NRG1AG1, or for the presence of a particular splicing variant encoded by NRG1AG1. An alteration in expression of a polypeptide encoded by NRG1AG1 can be, for example, an alteration in the quantitative polypeptide expression (i.e., the amount of polypeptide produced); an alteration in the composition of a polypeptide encoded by NRG1AG1 is an alteration in the qualitative polypeptide expression (e.g., expression of a mutant NRG1AG1 polypeptide or of a different splicing variant). In a preferred embodiment, diagnosis of a susceptibility to schizophrenia is made by detecting a particular splicing variant encoded by NRG1AG1, or a particular pattern of splicing variants.

Both quantitative and qualitative alterations can also be present. An “alteration” in the polypeptide expression or composition, as used herein, refers to an alteration in expression or composition in a test sample, as compared with the expression or composition of polypeptide by NRG1AG1 in a control sample. A control sample is a sample that corresponds to the test sample (e.g., is from the same type of cells), and is from an individual who is not affected by schizophrenia. An alteration in the expression or composition of the polypeptide in the test sample, as compared with the control sample, is indicative of a susceptibility to schizophrenia. Similarly, the presence of one or more different splicing variants in the test sample, or the presence of significantly different amounts of different splicing variants in the test sample, as compared with the control sample, is indicative of a susceptibility to schizophrenia. Various means of examining expression or composition of the polypeptide encoded by NRG1AG1 can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by a mutant NRG1AG1, or an antibody that specifically binds to a polypeptide encoded by a non-mutant gene, or an antibody that specifically binds to a particular splicing variant encoded by NRG1AG1, can be used to identify the presence in a test sample of a particular splicing variant or of a polypeptide encoded by a polymorphic or mutant NRG1AG1, or the absence in a test sample of a particular splicing variant or of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic for a susceptibility to schizophrenia, as is the presence (or absence) of particular splicing variants encoded by the NRG1AG1 gene.

In one embodiment of this method, the level or amount of polypeptide encoded by NRG1AG1 in a test sample is compared with the level or amount of the polypeptide encoded by NRG1AG1 in a control sample. A level or amount of the polypeptide in the test sample that is higher or lower than the level or amount of the polypeptide in the control sample, such that the difference is statistically significant, is indicative of an alteration in the expression of the polypeptide encoded by NRG1AG1, and is diagnostic for a susceptibility to schizophrenia. Alternatively, the composition of the polypeptide encoded by NRG1AG1 in a test sample is compared with the composition of the polypeptide encoded by NRG1AG1 in a control sample. A difference in the composition of the polypeptide in the test sample, as compared with the composition of the polypeptide in the control sample (e.g., the presence of different splicing variants), is diagnostic for a susceptibility to schizophrenia. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to schizophrenia.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to mutant or to non-mutant (native) NRG1AG1 polypeptide (e.g., to SEQ ID NO: 6, 7, 8, and/or 9), means for amplification of nucleic acids comprising NRG1AG1, or means for analyzing the nucleic acid sequence of NRG1AG1 or for analyzing the amino acid sequence of an NRG1 AG1 polypeptide, etc.

Screening Assays and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of SEQ ID NO: 1 or the complement of SEQ ID NO: 1, or a nucleic acid encoding an amino acid having the sequence of SEQ ID NO: 6, 7, 8, or 9, or a fragment or variant of such nucleic acids), under high stringency conditions as described above, and then assessing the sample for the presence (or absence) of hybridization. In a preferred embodiment, the high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a neuregulin 1-associated gene 1 nucleic acid), and the contacted sample is assessed for the presence or absence of hybridization. In a preferred embodiment, the nucleic acid containing a contiguous nucleotide sequence is completely complementary to a part of the nucleic acid molecule of interest.

In any of these embodiment, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes) which alter (e.g., increase or decrease) the activity of the polypeptides described herein, or which otherwise interact with the polypeptides herein. For example, such agents can be agents which bind to polypeptides described herein (e.g., NGR1AG1 binding agents); which have a stimulatory or inhibitory effect on, for example, activity of polypeptides of the invention; which change (e.g., enhance or inhibit) the ability of the polypeptides of the invention to interact with NRG1AG1 binding agents (e.g., receptors or other binding agents); or which alter posttranslational processing of the NRG1AG1 polypeptide (e.g., agents that alter proteolytic processing to direct the polypeptide from where it is normally synthesized to another location in the cell, such as the cell surface; agents that alter proteolytic processing such that more active polypeptide is released from the cell, etc.).

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des., 12:145).

In one embodiment, to identify agents which alter the activity of an NRG1AG1 polypeptide, a cell, cell lysate, or solution containing or expressing an NRG1AG1 polypeptide (e.g., SEQ ID NO: 6, 7, 8, or 9, or another splicing variant encoded by NRG1AG1), or a fragment or derivative thereof (as described above), can be contacted with an agent to be tested; alternatively, the polypeptide can be contacted directly with the agent to be tested. The level (amount) of NRG1AG1 activity is assessed (e.g., the level (amount) of NRG1AG1 activity is measured, either directly or indirectly), and is compared with the level of activity in a control (i.e., the level of activity of the NRG1AG1 polypeptide or fragment or derivative thereof in the absence of the agent to be tested). If the level of the activity in the presence of the agent differs, by an amount that is statistically significant, from the level of the activity in the absence of the agent, then the agent is an agent that alters the activity of NRG1AG1 polypeptide. An increase in the level of NRG1AG1 activity relative to a control, indicates that the agent is an agent that enhances (is an agonist of) NRG1AG1 activity. Similarly, a decrease in the level of NRG1AG1 activity relative to a control, indicates that the agent is an agent that inhibits (is an antagonist of) NRG1AG1 activity. In another embodiment, the level of activity of an NRG1AG1 polypeptide or derivative or fragment thereof in the presence of the agent to be tested, is compared with a control level that has previously been established. A level of the activity in the presence of the agent that differs from the control level by an amount that is statistically significant indicates that the agent alters NRG1AG1 activity.

The present invention also relates to an assay for identifying agents which alter the expression of the NRG1AG1 gene (e.g., antisense nucleic acids, fusion proteins, polypeptides, peptidomimetics, prodrugs, receptors, binding agents, antibodies, small molecules or other drugs, or ribozyrnes) which alter (e.g., increase or decrease) expression (e.g., transcription or translation) of the gene or which otherwise interact with the nucleic acids described herein, as well as agents identifiable by the assays. For example, a solution containing a nucleic acid encoding NRG1AG1 polypeptide (e.g., NRG1AG1 gene) can be contacted with an agent to be tested. The solution can comprise, for example, cells containing the nucleic acid or cell lysate containing the nucleic acid; alternatively, the solution can be another solution which comprises elements necessary for transcription/translation of the nucleic acid. Cells not suspended in solution can also be employed, if desired. The level and/or pattern of NRG1AG1 expression (e.g., the level and/or pattern of mRNA or of protein expressed, such as the level and/or pattern of different splicing variants) is assessed, and is compared with the level and/or pattern of expression in a control (i.e., the level and/or pattern of the NRG1AG1 expression in the absence of the agent to be tested). If the level and/or pattern in the presence of the agent differs, by an amount or in a manner that is statistically significant, from the level and/or pattern in the absence of the agent, then the agent is an agent that alters the expression of NRG1AG1. Enhancement of NRG1AG1 expression indicates that the agent is an agonist of NRG1AG1 activity. Similarly, inhibition of NRG1AG1 expression indicates that the agent is an antagonist of NRG1AG1 activity. In another embodiment, the level and/or pattern of NRG1AG1 polypeptide(s) (e.g., different splicing variants) in the presence of the agent to be tested, is compared with a control level and/or pattern that has previously been established. A level and/or pattern in the presence of the agent that differs from the control level and/or pattern by an amount or in a manner that is statistically significant indicates that the agent alters NRG1AG1 expression.

In another embodiment of the invention, agents which alter the expression of the NRG1AG1 gene or which otherwise interact with the nucleic acids described herein, can be identified using a cell, cell lysate, or solution containing a nucleic acid encoding the promoter region of the NRG1AG1 gene operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of NRG1AG1, as indicated by its ability to alter expression of a gene that is operably linked to the NRG1AG1 gene promoter. Enhancement of the expression of the reporter indicates that the agent is an agonist of NRG1AG1 activity. Similarly, inhibition of the expression of the reporter indicates that the agent is an antagonist of NRG1AG1 activity. In another embodiment, the level of expression of the reporter in the presence of the agent to be tested, is compared with a control level that has previously been established. A level in the presence of the agent that differs from the control level by an amount or in a manner that is statistically significant indicates that the agent alters NRG1AG1 expression.

Agents which alter the amounts of different splicing variants encoded by NRG1AG1 (e.g., an agent which enhances activity of a first splicing variant, and which inhibits activity of a second splicing variant), as well as agents which are agonists of activity of a first splicing variant and antagonists of activity of a second splicing variant, can easily be identified using these methods described above.

In other embodiments of the invention, assays can be used to assess the impact of a test agent on the activity of an NRG1AG1 polypeptide in relation to an NRG1AG1 binding agent. For example, a cell that expresses a compound that interacts with NRG1AG1 (herein referred to as a “NRG1AG1 binding agent”, which can be a polypeptide or other molecule that interacts with NRG1AG1, such as a receptor) is contacted with NRG1AG1 in the presence of a test agent, and the ability of the test agent to alter the interaction between NRG1AG1 and the NRG1AG1 binding agent is determined. Alternatively, a cell lysate or a solution containing the NRG1AG1 binding agent, can be used. An agent which binds to NRG1AG1 or the NRG1AG1 binding agent can alter the interaction by interfering with, or enhancing the ability of NRG1AG1 to bind to, associate with, or otherwise interact with the NRG1AG1 binding agent. Determining the ability of the test agent to bind to NRG1AG1 or an NRG1AG1 binding agent can be accomplished, for example, by coupling the test agent with a radioisotope or enzymatic label such that binding of the test agent to the polypeptide can be determined by detecting the labeled with ¹²⁵I, ³⁵S, ¹⁴C, or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with NRG1AG1 or an NRG1AG1 binding agent without the labeling of either the test agent, NRG1AG1, or the NRG1AG1 binding agent. McConnell, H. M. et al (1992) Science, 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

In another embodiment of the invention, assays can be used to identify polypeptides that interact with one or more NRG1AG1 polypeptides, as described herein. For example, a yeast two-hybrid system such as that described by Fields and Song (Fields, S. and Song, O., Nature 340:245-246 (1989)) can be used to identify polypeptides that interact with one or more NRG1AG1 polypeptides. In such a yeast two-hybrid system, vectors are constructed based on the flexibility of a transcription factor which has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also an NRG1AG1 polypeptide, splicing variant, or fragment or derivative thereof, and a second vector is used which includes a nucleic acid encoding a transcription activation domain and also a nucleic acid encoding a polypeptide which potentially may interact with the NRG1AG1 polypeptide, splicing variant, or fragment or derivative thereof (e.g., a NRG1AG1 polypeptide binding agent or receptor). Incubation of yeast containing the first vector and the second vector under appropriate conditions (e.g., mating conditions such as used in the Matchmaker™ system from Clontech) allows identification of colonies which express the markers of interest. These colonies can be examined to identify the polypeptide(s) which interact with the NRG1AG1 polypeptide or fragment or derivative thereof. Such polypeptides may be useful as agents which alter the activity of expression of an NRG1AG1 polypeptide, as described above.

In more than one embodiment of the above assay methods of the present invention, it maybe desirable to immobilize either NRG1AG1, the NRG1AG1 binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided which adds a domain that allows NRG1AG1 or an NRG1AG1 binding agent to be bound to a matrix or other solid support.

In another embodiment, modulators of expression of nucleic acid molecules of the invention are identified in a method wherein a cell, cell lysate, or solution containing a nucleic acid encoding NRG1AG1 is contacted with a test agent and the expression of appropriate mNRA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mNRA or polypeptide is less (statistically significantly less) in the presence of the test agent than in its absence, the test agent is identified as an inhibitor of the mRNA or polypeptide expression. The level of mNRA or polypeptide expression in the cells can be determined by methods described herein for detecting mNRA or polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein. In addition, an agent identified as described herein can be used to alter activity of a polypeptide encoded by neuregulin 1-associated gene 1, or to alter expression of neuregulin 1-associated gene 1, by contacting the polypeptide or the gene (or contacting a cell comprising the polypeptide or the gene) with the agent identified as described herein.

Pharmaceutical Compositions

The present invention also pertains to pharmaceutical compositions comprising nucleic acids described herein, particularly nucleotides encoding the polypeptides described herein; comprising polypeptides described herein (e.g., one or more of SEQ ID NO: 6, 7, 8, and/or 9, and/or other splicing variants encoded by NRG1AG1); and/or comprising an agent that alters (e.g., enhances or inhibits) NRG1AG1 gene expression or NRG1AG1 polypeptide activity as described herein. For instance, a polypeptide, protein (e.g., an NRG1AG1 receptor), fragment, fusion protein or prodrug thereof, or a nucleotide or nucleic acid construct (vector) comprising a nucleotide of the present invention, an agent that alters NRG1AG1 polypeptide activity, an agent that alters NRG1AG1 gene expression, or an NRG1AG1 binding agent or binding partner, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxyrnethylcellulose, polyvinyl pyro-lidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrol-lidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of schizophrenia, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Methods of Therapy

The present invention also pertains to methods of treatment (prophylactic and/or therapeutic) for schizophrenia, using an NRG1AG1 therapeutic agent. An “NRG1AG1 therapeutic agent” is an agent that alters (e.g., enhances or inhibits) NRG1AG1 polypeptide activity and/or NRG1AG1 gene expression, as described herein (e.g., an NRG1AG1 agonist or antagonist). NRG1AG1 therapeutic agents can alter NRG1AG1 polypeptide activity or gene expression by a variety of means, such as, for example, by providing additional NRG1AG1 polypeptide or by upregulating the transcription or translation of the NRG1AG1 gene; by altering posttranslational processing of the NRG1AG1 polypeptide; by altering transcription of NRG1AG1 splicing variants; or by interfering with NRG1AG1 polypeptide activity (e.g., by binding to an NRG1AG1 polypeptide), or by downregulating the transcription or translation of the NRG1AG1 gene. Representative NRG1AG1 therapeutic agents include the following:

nucleic acids or fragments or derivatives thereof described herein, particularly nucleotides encoding the polypeptides described herein and vectors comprising such nucleic acids (e.g., a gene, cDNA, and/or mNRA, such as a nucleic acid encoding an NRG1AG1 polypeptide or active fragment or derivative thereof, or an oligonucleotide; for example, SEQ ID NO: 1 or a nucleic acid encoding SEQ ID NO: 6, 7, 8, or 9, or fragments or derivatives thereof);

polypeptides described herein (e.g., one or more of SEQ ID NO: 6, 7, 8, and/or 9, and/or other splicing variants encoded by NRG1AG1, or fragments or derivatives thereof);

other polypeptides (e.g., NRG1AG1 receptors); NRG1AG1 binding agents; peptidomimetics; fusion proteins or prodrugs thereof, antibodies (e.g., an antibody to a mutant NRG1AG1 polypeptide, or an antibody to a non-mutant NRG1AG1 polypeptide, or an antibody to a particular splicing variant encoded by NRG1AG1, as described above); ribozymes; other small molecules;

and other agents that alter (e.g., enhance or inhibit) NRG1AG1 gene expression or polypeptide activity, that alter posttranslational processing of the NRG1AG1 polypeptide, or that regulate transcription of NRG1AG1 splicing variants (e.g., agents that affect which splicing variants are expressed, or that affect the amount of each splicing variant that is expressed).

In a preferred embodiment, the NRG1AG1 therapeutic agent is a nucleic acid encoding one or more NRG1AG1 polypeptides (e.g., encoding SEQ ID NO: 6, 7, 8, and/or 9, or a fragment or derivative thereof); in another preferred embodiment, the NRG1AG1 therapeutic agent is a nucleic acid comprising a fragment of the NRG1AG1 gene (e.g., comprising a fragment of SEQ ID NO: 1, or a derivative thereof), such as a regulatory region of the NRG1AG1 gene; in yet another preferred embodiment, the NRG1AG1 therapeutic agent is a nucleic acid comprising the NRG1AG1 gene regulatory region and also a nucleic acid encoding one or more NRG1AG1 polypeptides (or fragments or derivatives thereof).

More than one NRG1AG1 therapeutic agent can be used concurrently, if desired.

The NRG1AG1 therapeutic agent that is a nucleic acid is used in the treatment of schizophrenia. The term, “treatment” as used herein, refers not only to ameliorating symptoms associated with the disease, but also preventing or delaying the onset of the disease, and also lessening the severity or frequency of symptoms of the disease. The therapy is designed to alter (e.g., inhibit or enhance), replace or supplement activity of an NRG1AG1 polypeptide in an individual. For example, an NRG1AG1 therapeutic agent can be administered in order to upregulate or increase the expression or availability of the NRG1AG1 gene or of specific splicing variants of NRG1AG1, or, conversely, to downregulate or decrease the expression or availability of the NRG1AG1 gene or specific splicing variants of NRG1AG1. Upregulation or increasing expression or availability of a native NRG1AG1 gene or of a particular splicing variant could interfere with or compensate for the expression or activity of a defective gene or another splicing variant; downregulation or decreasing expression or availability of a native NRG1AG1 gene or of a particular splicing variant could minimize the expression or activity of a defective gene or the particular splicing variant and thereby minimize the impact of the defective gene or the particular splicing variant.

The NRG1AG1therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding an NRG1AG1 polypeptide, such as SEQ ID NO: 1; or another nucleic acid that encodes an NRG1AG1 polypeptide or a splicing variant, derivative or fragment thereof, such as a nucleic acid encoding SEQ ID NO: 6, 7, 8, and/or 9) can be used, either alone or in a pharmaceutical composition as described above. For example, NRG1AG1 or a cDNA encoding the NRG1AG1 polypeptide, either by itself or included within a vector, can be introduced into cells (either in vitro or in vivo) such that the cells produce native NRG1AG1 polypeptide. If necessary, cells that have been transformed with the gene or cDNA or a vector comprising the gene or CDNA can be introduced (or re-introduced) into an individual affected with the disease. Thus, cells which, in nature, lack native NRG1AG1 expression and activity, or have mutant NRG1AG1 expression and activity, or have expression of a disease-associated NRG1AG1 splicing variant, can be engineered to express NRG1AG1 polypeptide or an active fragment of the NRG1AG1 polypeptide (or a different variant of NRG1AG1 polypeptide). In a preferred embodiment, nucleic acid encoding the NRG1AG1 polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) which specifically hybridizes to the mRNA and/or genomic DNA of NRG1AG1 is administered or generated in situ. The antisense nucleic acid that specifically hybridizes to the mRNA and/or DNA inhibits expression of the NRG1AG1 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA which is complementary to a portion of the rnRNA and/or DNA which encodes NRG1AG1 polypeptide. Alternatively, the antisense construct can be an oligonucleotide probe which is generated ex vivo and introduced into cells; it then inhibits expression by hybridizing with the mNRA and/or genomic DNA of NRG1AG1. In one embodiment, the oligonucleotide probes are modified oligonucleotides which are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al. ( (1988) Cancer Res 48:2659-2668). With respect to antisense DNA, oligodeoxyribonucleotides derived from the translation initiation site, e.g. between the −10 and +10 regions of NRG1AG1 sequence, are preferred.

To perform antisense therapy, oligonucleotides (mNRA, cDNA or DNA) are designed that are complementary to mNRA encoding NRG1AG1. The antisense oligonucleotides bind to NRG1AG1 mRNA transcripts and prevent translation. Absolute complementarity, although preferred, is not required. a sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al. (1989) Proc. Natl. Acad. Sci. USA 86:6553-6556; Lemaitre et al, (1987), Proc. Natl. Acad Sci. USA 84:648-652; PCT International Publication No. W088/09810) or the blood-brain barrier (see, e.g., PCT International Publication No. W089/10134), or hybridization-triggered cleavage agents (see, e.g., Krol et al. (1988) BioTechniques 6:958-976) or intercalating agents. (See, e.g., Zon, (1988), Pharm. Res. 5:539-549). To this end, the oligonucleotide maybe conjugated to another molecule (e.g., a peptide, hybridization triggered cross-linking agent, transport agent, hybridization-triggered cleavage agent).

The antisense molecules are delivered to cells which express NRG1AG1 in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous NRG1AG1 transcripts and thereby prevent translation of the NRG1AG1 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Endogenous NRG1AG1 expression can also be reduced by inactivating or “knocking out” NRG1AG1 or its promoter using targeted homologous recombination (e.g., see Smithies et al. (1985) Nature 317:230-234; Thomas & Capecchi (1987) Cell 51:503-512; Thompson et al. (1989) Cell 5:313-321). For example, a mutant, non-functional NRG1AG1 (or a completely unrelated DNA sequence) flanked by DNA homologous to the endogenous NRG1AG1 (either the coding regions or regulatory regions of NRG1AG1) can be used, with or without a selectable marker and/or a negative selectable marker, to transfect cells that express NRG1AG1 in vivo. Insertion of the DNA construct, via targeted homologous recombination, results in inactivation of NRG1AG1. The recombinant DNA constructs can be directly administered or targeted to the required site in vivo using appropriate vectors, as described above. Alternatively, expression of non-mutant NRG1AG1 can be increased using a similar method: targeted homologous recombination can be used to insert a DNA construct comprising a non-mutant, functional NRG1AG1 (e.g., a gene having SEQ ID NO: 1), or a portion thereof, in place of a mutant NRG1AG1 in the cell, as described above. In another embodiment, targeted homologous recombination can be used to insert a DNA construct comprising a nucleic acid that encodes an NRG1AG1 polypeptide variant that differs from that present in the cell.

Alternatively, endogenous NRG1AG1 expression can be reduced by targeting deoxyribonucleotide sequences complementary to the regulatory region of NRG1AG1 (i.e., the NRG1AG1 promoter and/or enhancers) to form triple helical structures that prevent transcription of NRG1AG1 in target cells in the body. (See generally, Helene, C. (1991) Anticancer Drug Des., 6(6):569-84; Helene, C., et al. (1992) Ann, N.Y. Acad. Sci., 660:27-36; and Maher, L. J. (1992) Bioassays 14(12):807-15). Likewise, the antisense constructs described herein, by antagonizing the normal biological activity of one of the NRG1AG1 proteins, can be used in the manipulation of tissue, e.g. tissue differentiation, both in vivo and for ex vivo tissue cultures. Furthermore, the anti-sense techniques (e.g. microinjection of antisense molecules, or transfection with plasmids whose transcripts are anti-sense with regard to an NRG1AG1 mRNA or gene sequence) can be used to investigate role of NRG1AG1 in developmental events, as well as the normal cellular function of NRG1AG1 in adult tissue. Such techniques can be utilized in cell culture, but can also be used in the creation of transgenic animals.

In yet another embodiment of the invention, other NRG1AG1 therapeutic agents as described herein can also be used in the treatment or prevention of schizophrenia. The therapeutic agents can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-mutant NRG1AG1 polypeptide in conjunction with antisense therapy targeting mutant NRG1AG1 mRNA; administration of a first splicing variant encoded by NRG1AG1 in conjunction with antisense therapy targeting a second splicing variant encoded by NRG1AG1), can also be used.

The invention will be further described by the following non-limiting examples. The teachings of all publications cited herein are incorporated herein by reference in their entirety.

### EXEMPLIFICATION

### Identification of Gene With Linkage to Schizophrenia

Patient Population

The lifetime expectancy of schizophrenia in Iceland is similar to what has been observed in the neighboring countries, 0.6% for males and 0.9% for females. A team of seven psychiatrists who diagnose patients and confirm the diagnosis of previously diagnosed schizophrenics and collect samples was employed. Each psychiatrist interviewed, using the Schedule for Schizophrenia and Affective Disorders, lifetime version (SADS-L) (Endicott, J. and Spitzer, R. L., Arch. Gen. Psychiatry 35:837 (1978)). The information from the SADS-L interviews was then used to classify all cases in accordance with research diagnostic criteria (RDC) and the Diagnosis and Statistical Manual of Mental Disorders, third edition, revised (DMS III-R). Furthermore, the operational criteria OPCRIT checklist for psychotic illness was also used to facilitate a polydiagnostic approach to psychotic illness (McGuffin, P. et al., Arch. Gen Psychiatry 48(8):764-70 (1991)).

Construction of a BAC Contig

A BAC (bacterial artificial chromosome) contig for the region of interest was generated using the RCPI11 Human BAC library (Pieter dejong, Roswell Park). BACs were identified by hybridization using available STS markers and microsatellite markers in the region, followed by successive rounds of hybridization using markers designed from BAC end sequences. Hybridization results were confirmed and the order of the BACs determined by PCR using all available markers in the region. The primary goal was to achieve a high resolution ordering of the microsatellite markers.

Search for New Microsatellite Markers

BACs were shotgun cloned and gridded onto membranes. Clones containing microsatellite repeats were identified by hybridization with oligonucleotide probes consisting of microsatellite repeat sequences. Positive clones were analyzed by sequencing and primers designed to amplify the microsatellites.

DNA Sequencing

Nine BACs, covering the minimum tiling path of the region of interest, were analyzed by shotgun cloning and sequencing. Dye terminator (ABI PRISM BigDye™) chemistry was used for fluorescent automated DNA sequencing. ABI prism 377 sequencers were used to collect data and the Phred/Phrap/Consed software package in combination with the Polyphred software used to assemble sequences.

Search for Exons in Sequence Databases

Exons/Genes were Searched for by BLAST Alignment to DNA and Protein Databases.

Search for New Exons in cDNA Libraries

Both 3′ and 5′ RACE (rapid amplification of cDNA ends) was carried out using the Marathon-ReadyTM cDNA from Clontech laboratories Inc and cDNA libraries made at deCODE genetics. cDNA libraries from whole brain, fetal brain and testis were used.

Search for New Exons Using Exon Prediction Tools

Gene miner software (deCODE genetics) was used to predict where exons were in our 1.5 Mb sequence. Primers for amplifying these candidate exons from cDNA libraries were designed, touch down PCRs were carried out, and the products were verified by sequencing.

Trapping Exons

Exons were “trapped” by using the Exon trapping kit from Live technologies. Primers were designed for amplifying these candidate exons from cDNA libraries, touch down PCRs were carried out, and the products were verified by sequencing.

Genome-Wide Scan

Samples from affected individuals related within 6 meiotic events, 260 affected individuals and 334 associated relatives, have been genotyped using a marker set of 950 microsatellite markers. One locus, 8p21-8p11, was reexamined with additional 150 follow-up markers. In addition to the 260 affected individuals and their relatives in the genome wide scan, 132 affected individuals and 147 available relatives were also genotyped using the 150 microsatellite markers for the 8p21-11 locus.

Statistical Analysis

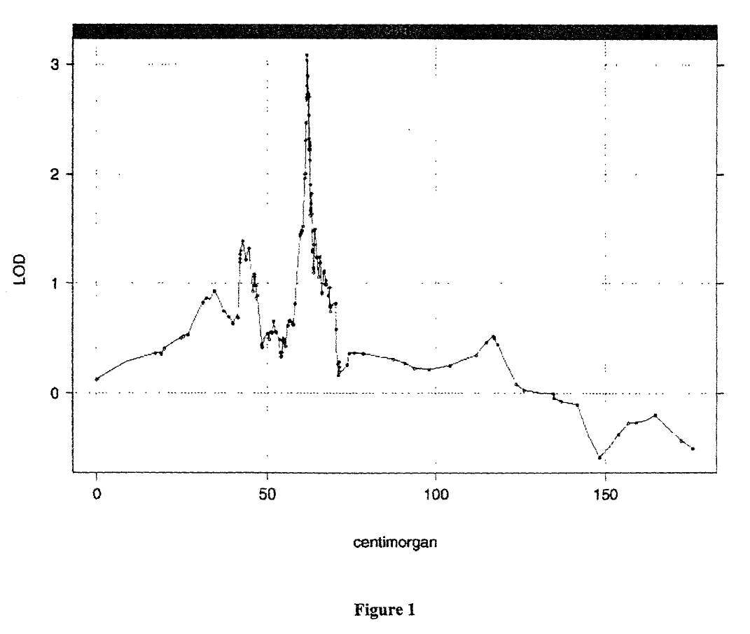

A linkage analysis was performed with the Allegro software. FIG. 1 displays the results for the Allele-Sharing Model using the CS affected pedigree (158 affected individuals, maximum distance of 5 meiotic events between affected individuals).

Physical Mapping of the Probable Schizophrenic Locus (Locus on 8p21-11)

The most significant locus that was found, with a maximum LOD score near 3, was physically mapped using bacterial artificial chromosomes (BACs). Initially the locus was wide, around 30 cM. Only a small fraction of this region had been sequenced previously, with the total cumulative number of bases of around 5 Mb. The published order of markers in the region was not correct and most of the polymorphic markers known in this region had not been radiation hybrid mapped. The primary goal with the BAC map was to achieve a high-resolution ordering (100 to 150 kb) of all polymorphic markers in this region and search for new polymorphic markers

By screening BAC libraries with primers from the region, 3000 BACs were retrieved by hybridization and PCR methods. Contig mapping was performed; 940 of these clones were assigned by PCR and hybridization to contigs. In addition, 252 additional BACs were assigned to contigs based on fingerprint analysis ( a total of 1192 BAC clones have been assigned to contigs). After correcting the marker order the maximum lod score is 3.1 (FIG. 1). The order of 534 markers in the 30 cM BAC area covered by the BAC contig has now been determined. The physical map has allowed the ordering and placement of polymorphic microsatellite markers and STS markers. BACs were subcloned from the BAC contig and searched for new microsatellites by hybridization. Samples were genotyped using, on average, a polymorphic microsatellite marker every 0.17 cM throughout the locus. Microsatellites are set forth in Appendix II.

As a result of the physical mapping effort the locus was narrowed to approximately 20 cM. This 20 cM region was spanned by four big contigs, 2-10 Mb each. The main peak extended over 7 cM and this region resided in one BAC contig. The four contigs were correctly ordered based on data from radiation hybrid mapped markers in these contigs, yeast artificial chromosomes (YAC) maps and by comparing haplotypes within families. Now that the marker order has been corrected, as described herein, the densely mapped markers can be used to reconstruct more correct haplotypes and search for at-risk haplotypes, haplotypes giving substantial overlap between families.

Identification of at-risk Haplotypes

Locus 8p21-11

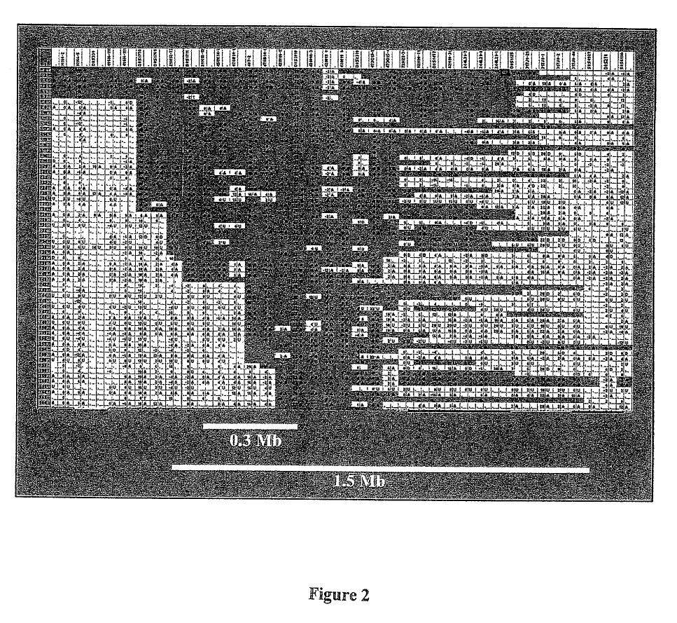

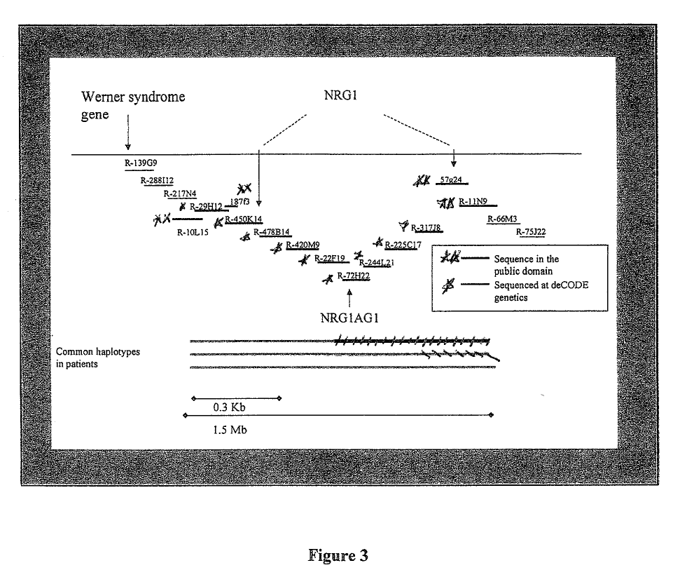

Using genotypes for the densely mapped markers, haplotypes of the affected individuals were constructed, and candidate at-risk haplotypes which are carried by three or more affected individuals within each individual family were identified. By comparing these candidate haplotypes across families, it was found that some of these haplotypes have substantial overlap (FIG. 2). The core of the haplotype found in affected individuals (6 markers telomeric to D8S1810, 0.3 Mb) was found in 10% of the patients (37 out of 746 chromosomes investigated). In comparison, 3% of controls had this haplotype (6 out of 376). FIG. 2 shows 44 patient haplotypes having a part of this at-risk haplotype. FIG. 3 shows an overview of the order of sequenced BACS and the boundaries for the at-risk haplotypes at locus 8p12.

The results from the linkage and haplotype analyses strongly suggested the presence of a disease-susceptibility gene residing in a 1.5 Mb segment at 8p12, harboring exons from the gene, neuregulin 1 (NRG1) and from a new gene Neuregulin-1-associated gene 1 (NRG1AG1). The gene for neuregulin 1 (NRG1) is described further in U.S. patent application Ser. No. 09/515,716, attorney docket no. 2345-2004-000, entitled “Human Schizophrenia Gene, ” and filed concurrently with the present application and incorporated herein by reference in its entirety.

The Sequence of the Candidate Region

Locus 8p12

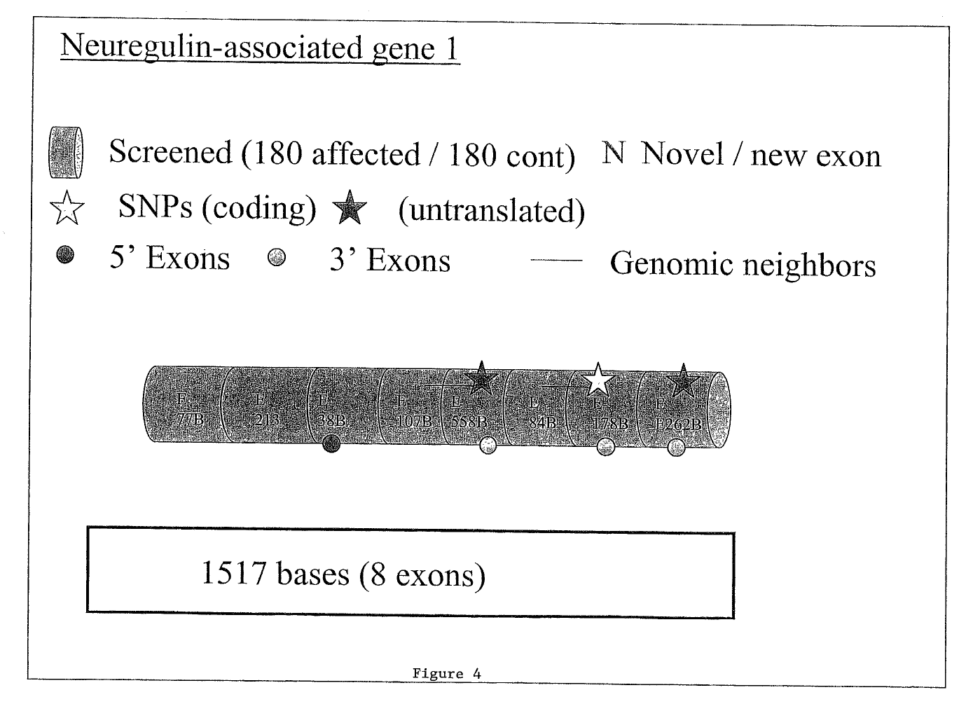

Sequencing of 1.5 Mb of the BAC contig on 8p12 where candidate haplotypes showed substantial overlap between families. This sequence was in one contig and harbors the gene, neuregulin-1-associated gene 1 (NRG1AG1). Eight exons from this gene were identified. The open reading frames showed no homology with other known genes. A depiction of the exons, single nucleotide polymorphisms (SNPs), and exons is presented in FIG. 4. Since this gene is within neuregulin-1 and located within the 1.5 Mb region defined by the at-risk haplotypes, it is a strong candidate gene for susceptibility to schizophrenia.

Neuregulin 1 (NRG1)

Neuregulin 1 (also called ARIA, GGF2 and heregulin) are a group of polypeptide factors that arise from alternative RNA splicing of a single gene (Fischbach, G. D. and Rosen, K. M., Annu. Rev. Neurosci. 20:429-458 (1997); Orr-Urtreger, A., et al., Proc. Natl Acad. Sci. USA 90:1746-1750 (1993); see also, Corfas, G. et al., Neuron 14(1):103-15 (1995) and Meyer, D. et al., Development 124(18):3575-86 (1997)). Neuregulin is expressed in many tissues, among others in the central nervous system (see, e.g., Corfas, G. et al., Neuron 14(1):103-115 (1995)). Neuregulin 1 gene is expected to be associated with schizophrenia for many reasons, including its role in the expression of the NMDA receptor, in activation of AChR gene expression as well as activation of epidermal growh factor receptors and GABA(a) receptor subunits, and also its induction of components in a G-protein signaling cascade. For a further discussion of these activities of neuregulin 1, see U.S. patent application Ser. No. 09/515,716, filed concurrently with the present application.

Neuregulin-1-associated gene 1 (NRG1AG1)

Neuregulin-1-associated gene 1 is a previously unknown gene. Eight exons were found for this gene (FIG. 4) and four alternatively spliced forms (see Appendix III). The open reading frames did not reveal any homology with known genes. This gene is within NRG1 It is much smaller than NRG1, having 112929 bases in its gene sequence. Not all exons have been found for this gene yet; a 5′ exon is still missing for two of the splice variants. It is likely that this gene is involved in the same processes as neuregulin 1, since it is within that gene and on the same strand.

This gene was identified by predicting where exons might be located in the 1.5 Mb sequence defined by the at-risk haplotypes. Primers were then designed, and cDNA libraries (Brain) were screened. A BLAST alignment was also carried out, BLASTing the 1.5 Mb sequence against the EST database. Two EST clones were in the EST database overlapping with this gene. IMAGE number and accession numbers for these clones are: IMAGE:727960 1 (AA394309, AA435550) and IMAGE 1643938 (AI027638).

Mutation Analysis

Neuregulin-1-associated gene 1 (8p12)

All eight exons have been searched in 180 patients and 180 control individuals. A number of SNPs have been found (FIG. 4; see also Appendix II). SNPs, deletions and insertions are shown in Appendix II.

Bacterial Artificial Clones (BACs)

The BAC clones R-217N4, R-29H12, R-450K14, R-478B14, R-420M9, R-22F19, R-72H22, R-244L21, R-225C17, R-317J8 and R-541C15 are from the RCP111 Human BAC library (Pieter dejong, Roswell Park). The vector used was pBACe3.6. The clones were picked into a 94 well microtiter plate containing LB/chloramphenicol (25 μg/ml)/glycerol (7.5%) and stored at −80° C. after a single colony has been positively identified through sequencing. The clones can then be streaked out on a LB agar plate with the appropriate antibiotic, chloramphenicol (25 μg/ml)/sucrose (5%).

While this invention has been particularly shown and described with reference to preferred embodiments thereof, it will be understood by those skilled in the art that various changes in form and details may be made therein without departing from the spirit and scope of the invention as defined by the appended claims.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. An isolated nucleic acid molecule comprising a neuregulin 1-associated gene 1, or a fragment or variant thereof.

2. The isolated nucleic acid molecule of claim 1, wherein the neuregulin 1-associated gene 1 has the nucleotide sequence of SEQ ID NO: 1.

3. A nucleic acid encoding a polypeptide having an amino acid sequence selected from the group consisting of SEQ ID NO: 6, SEQ ID NO: 7, SEQ ID NO: 8 and SEQ ID NO: 9.

4. An isolated nucleic acid molecule comprising a nucleotide sequence selected from the group consisting of SEQ ID NO: 1 and the complement of SEQ ID NO: 1.

5. An isolated nucleic acid molecule which hybridizes under high stringency conditions to a nucleotide sequence selected from the group consisting of SEQ ID NO: 1 and the complement of SEQ ID NO: 1.

6. An isolated nucleic acid molecule which hybridizes under high stringency conditions to a nucleotide sequence encoding an amino acid sequence selected from the group consisting of: SEQ ID NO: 6, SEQ ID NO: 7, SEQ ID NO: 8, and SEQ ID NO: 9.

7. A method for assaying the presence of a first nucleic acid molecule in a sample, comprising contacting said sample with a second nucleic acid molecule comprising a nucleotide sequence selected from the group consisting of SEQ ID NO: 1 and the complement of SEQ ID NO: 1, under high stringency conditions.

8. A vector comprising an isolated nucleic acid molecule selected from the group consisting of: SEQ ID NO: 1, the complement of SEQ ID NO: 1, a nucleic acid encoding SEQ ID NO: 6, a nucleic acid encoding SEQ ID NO: 7, a nucleic acid encoding SEQ ID NO: 8, and a nucleic acid encoding SEQ ID NO: 9, operatively linked to a regulatory sequence.

9. A recombinant host cell comprising the vector of claim 8.

10. A method for producing a polypeptide encoded by an isolated nucleic acid molecule, comprising culturing the recombinant host cell of claim 9 under conditions suitable for expression of said nucleic acid molecule.

11. An isolated polypeptide encoded by an neuregulin 1-associated gene 1, or a fragment or variant of said polypeptide.

12. The isolated polypeptide of claim 11, wherein the neuregulin 1-associated gene 1 has the sequence of SEQ ID NO: 1 or the complement of SEQ ID NO: 1.

13. The isolated polypeptide of claim 11, wherein the polypeptide has an amino acid sequence selected from the group consisting of SEQ ID NO: 6, SEQ ID NO: 7, SEQ ID NO: 8 and SEQ ID NO: 9.

14. An isolated polypeptide comprising an amino acid sequence which is greater than about 90 percent identical to an amino acid sequence selected from the group consisting of SEQ ID NO: 6, SEQ ID NO: 7, SEQ ID NO: 8 and SEQ ID NO: 9.

15. A fusion protein comprising an isolated polypeptide of claim 11.

16. An antibody, or an antigen-binding fragment thereof, which selectively binds to a polypeptide of claim 11.

17. An antibody, or an antigen-binding fragment thereof, which selectively binds to an amino acid sequence selected from the group consisting of SEQ ID NO: 6, SEQ ID NO: 7, SEQ ID NO: 8 and SEQ ID NO: 9, or to a fragment or variant of said amino acid sequence.

18. A method for assaying the presence of a polypeptide encoded by an isolated nucleic acid molecule according to claim 1 in a sample, comprising contacting said sample with an antibody which specifically binds to the encoded polypeptide.

19. A method of diagnosing a susceptibility to schizophrenia in an individual, comprising detecting a polymorphism in neuregulin 1-associated gene 1, wherein the presence of the polymorphism in the gene is indicative of a susceptibility to schizophrenia.

20. A method of diagnosing a susceptibility to schizophrenia, comprising detecting an alteration in the expression or composition of a polypeptide encoded by neuregulin 1-associated gene 1 in a test sample, in comparison with the expression or composition of a polypeptide encoded by neuregulin 1-associated gene 1 in a control sample, wherein the presence of an alteration in expression or composition of the polypeptide in the test sample is indicative of a susceptibility to schizophrenia.

21. The method of claim 20, wherein the alteration in the expression or composition of a polypeptide encoded by neuregulin 1-associated gene 1 comprises expression of a splicing variant polypeptide in a test sample that differs from a splicing variant polypeptide expressed in a control sample.

22. A method of identifying an agent which alters activity of a polypeptide of claim 11, comprising:
a) contacting the polypeptide or a derivative or fragment thereof, with an agent to be tested;
b) assessing the level of activity of the polypeptide or derivative or fragment thereof; and
c) comparing the level of activity with a level of activity of the polypeptide or active derivative or fragment thereof in the absence of the agent,
wherein if the level of activity of the polypeptide or derivative or fragment thereof in the presence of the agent differs, by an amount that is statistically significant, from the level in the absence of the agent, then the agent is an agent that alters activity of the polypeptide.

23. An agent which alters activity of a polypeptide encoded by neuregulin 1-associated gene 1, identifiable according to the method of claim 22.

24. An agent which alters activity of a polypeptide encoded by neuregulin 1-associated gene 1, wherein the agent is selected from the group consisting of: a neuregulin 1-associated gene 1 receptor; a neuregulin 1-associated gene 1 binding agent; a peptidomimetic; a fusion protein; a prodrug; an antibody; and a ribozyme.

25. A method of altering activity of a polypeptide encoded by neuregulin 1-associated gene 1, comprising contacting the polypeptide with an agent of claim 24.

26. A method of identifying an agent which alters interaction of the polypeptide of claim 11 with a neuregulin 1-associated gene 1 binding agent, comprising:
a) contacting the polypeptide or a derivative or fragment thereof, the binding agent and with an agent to be tested;
b) assessing the interaction of the polypeptide or derivative or fragment thereof with the binding agent; and
c) comparing the level of interaction with a level of interaction of the polypeptide or derivative or fragment thereof with the binding agent in the absence of the agent,
wherein if the level of interaction of the polypeptide or derivative or fragment thereof in the presence of the agent differs, by an amount that is statistically significant, from the level of interaction in the absence of the agent, then the agent is an agent that alters interaction of the polypeptide with the binding agent.

27. An agent which alters interaction of a neuregulin 1-associated gene 1 polypeptide with a neuregulin 1-associated gene 1 binding agent, identifiable according to the method of claim 26.

28. An agent which alters interaction of a neuregulin 1-associated gene 1 polypeptide with a first neuregulin 1-associated gene 1 binding agent, selected from the group consisting of: a neuregulin 1-associated gene 1 receptor; a second neuregulin 1-associated gene 1 binding agent; a peptidomimetic; a fusion protein; a prodrug; an antibody; and a ribozyme.

29. A method of altering interaction of a neuregulin 1-associated gene 1 polypeptide with a neuregulin 1-associated gene 1 binding agent, comprising contacting the neuregulin 1-associated gene 1 polypeptide and/or the neuregulin 1-associated gene 1 binding agent with an agent of claim 28.

30. A method of identifying an agent which alters expression of neuregulin 1-associated gene 1, comprising the steps of:
a) contacting a solution containing a nucleic acid of claim 1 or a derivative or fragment thereof with an agent to be tested;
b) assessing the level of expression of the nucleic acid, derivative or fragment; and
C) comparing the level of expression with a level of expression of the nucleic acid, derivative or fragment in the absence of the agent,
wherein if the level of expression of the nucleotide, derivative or fragment in the presence of the agent differs, by an amount that is statistically significant, from the expression in the absence of the agent, then the agent is an agent that alters expression of neuregulin 1-associated gene 1.

31. An agent which alters expression of neuregulin 1-associated gene 1, identifiable according to the method of claim 30.

32. A method of identifying an agent which alters expression of neuregulin 1-associated gene 1, comprising the steps of:
a) contacting a solution containing a nucleic acid comprising the promoter region of neuregulin 1-associated gene 1 operably linked to a reporter gene, with an agent to be tested;
b) assessing the level of expression of the reporter gene; and
c) comparing the level of expression with a level of expression of the reporter gene in the absence of the agent,
wherein if the level of expression of the reporter gene in the presence of the agent differs, by an amount that is statistically significant, from the level of expression in the absence of the agent, then the agent is an agent that alters expression of neuregulin 1-associated gene 1.

33. An agent which alters expression of neuregulin 1-associated gene 1, identifiable according to the method of claim 32.

34. A method of identifying an agent which alters expression of neuregulin 1-associated gene 1, comprising the steps of:
a) contacting a solution containing a nucleic acid of claim 1 or a derivative or fragment thereof with an agent to be tested;
b) assessing expression of the nucleic acid, derivative or fragment; and
c) comparing expression with expression of the nucleic acid, derivative or fragment in the absence of the agent,
wherein if expression of the nucleotide, derivative or fragment in the presence of the agent differs, by an amount that is statistically significant, from the expression in the absence of the agent, then the agent is an agent that alters expression of neuregulin 1-associated gene 1.

35. The method of claim 34, wherein the expression of the nucleotide, derivative or fragment in the presence of the agent comprises expression of one or more splicing variant(s) that differ in kind or in quantity from the expression of one or more splicing variant(s) the absence of the agent.

36. An agent which alters expression of neuregulin 1-associated gene 1, identifiable according to the method of claim 34.

37. An agent which alters expression of neuregulin 1-associated gene 1, selected from the group consisting of: antisense nucleic acid to neuregulin 1-associated gene 1; a neuregulin 1-associated gene 1 polypeptide; a neuregulin 1-associated gene 1 receptor; a neuregulin 1-associated gene 1 binding agent; a peptidomimetic; a fusion protein; a prodrug thereof, an antibody; and a ribozyme.

38. A method of altering expression of neuregulin 1-associated gene 1, comprising contacting a cell containing neuregulin 1-associated gene 1 with an agent of claim 37.

39. A method of identifying a polypeptide which interacts with a neuregulin 1-associated gene 1 polypeptide, comprising employing a two yeast hybrid system using a first vector which comprises a nucleic acid encoding a DNA binding domain and a neuregulin 1-associated gene 1 polypeptide, splicing variant, or fragment or derivative thereof, and a second vector which comprises a nucleic acid encoding a transcription activation domain and a nucleic acid encoding a test polypeptide, wherein if transcriptional activation occurs in the two yeast hybrid system, the test polypeptide is a polypeptide which interacts with a neuregulin 1-associated gene polypeptide.

40. A neuregulin 1-associated gene 1 therapeutic agent selected from the group consisting of: a neuregulin 1-associated gene 1 or fragment or derivative thereof; a polypeptide encoded by neuregulin 1-associated gene 1 ;a neuregulin 1-associated gene 1 receptor; a neuregulin 1-associated gene 1 binding agent; a peptidomimetic; a fusion protein; a prodrug; an antibody; an agent that alters neuregulin 1-associated gene 1 expression; an agent that alters activity of a polypeptide encoded by neuregulin 1-associated gene 1; an agent that alters posttranscriptional processing of a polypeptide encoded by neuregulin 1-associated gene 1; an agent that alters interaction of a neuregulin 1-associated gene 1 with a neuregulin 1-associated gene 1 binding agent; an agent that alters transcription of splicing variants encoded by neuregulin 1-associated gene 1; and a ribozyme.

41. A pharmaceutical composition comprising a neuregulin 1-associated gene 1 therapeutic agent of claim 40.

42. The pharmaceutical composition of claim 41, wherein the neuregulin 1-associated gene 1 therapeutic agent is an isolated nucleic acid molecule comprising a neuregulin 1-associated gene 1 or fragment or derivative thereof.

43. The pharmaceutical composition of claim 41, wherein the neuregulin 1-associated gene 1 therapeutic agent is a polypeptide encoded by the neuregulin 1-associated gene 1.

44. A method of treating schizophrenia in an individual, comprising administering a neuregulin 1-associated gene 1 therapeutic agent to the individual, in a therapeutically effective amount.

45. The method of claim 44, wherein the neuregulin 1-associated gene 1 therapeutic agent is a neuregulin 1-associated gene 1 agonist.

46. The method of claim 45 wherein the neuregulin 1-associated gene 1 therapeutic agent is a neuregulin 1-associated gene 1 antagonist.

47. A transgenic animal comprising a nucleic acid selected from the group consisting of: an exogenous neuregulin 1-associated gene 1 and a nucleic acid encoding a neuregulin 1-associated gene 1 polypeptide.

48. A method for assaying a sample for the presence of a neuregulin 1-associated gene 1 nucleic acid, comprising:
a) contacting said sample with a nucleic acid comprising a contiguous nucleotide sequence which is at least partially complementary to a part of the sequence of said neuregulin 1-associated gene 1 nucleic acid under conditions appropriate for hybridization, and
b) assessing whether hybridization has occurred between a neuregulin 1-associated gene 1 nucleic acid and said nucleic acid comprising a contiguous nucleotide sequence which is at least partially complementary to a part of the sequence of said neuregulin 1-associated gene 1 nucleic acid.

49. The method of claim 48, wherein said nucleic acid comprising a contiguous nucleotide sequence is completely complementary to a part of the sequence of said neuregulin 1-associated gene 1 nucleic acid.

50. The method of claim 48, comprising amplification of at least part of said neuregulin 1-associated gene 1 nucleic acid.

51. The method of claim 48, wherein said contiguous nucleotide sequence is 100 or fewer nucleotides in length and is either: a) at least 80% identical to a contiguous sequence of nucleotides in SEQ ID NO: 1; b) at least 80% identical to the complement of a contiguous sequence of nucleotides in SEQ ID NO: 1; or c) capable of selectively hybridizing to said neuregulin 1-associated gene 1 nucleic acid.

52. A reagent for assaying a sample for the presence of a neuregulin 1-associated gene 1 nucleic acid, said reagent comprising a nucleic acid comprising a contiguous nucleotide sequence which is at least partially complementary to a part of the nucleotide sequence of said neuregulin 1-associated gene 1 nucleic acid.

53. The reagent of claim 52, wherein the nucleic acid comprises a contiguous nucleotide sequence which is completely complementary to a part of the nucleotide sequence of said neuregulin 1-associated gene 1 nucleic acid.

54. A reagent kit for assaying a sample for the presence of a neuregulin 1-associated gene 1 nucleic acid, comprising in separate containers:
a) one or more labeled nucleic acids comprising a contiguous nucleotide sequence which is at least partially complementary to a part of the nucleotide sequence of said neuregulin 1-associated gene 1 nucleic acid, and
b) reagents for detection of said label.

55. The reagent kit of claim 54, wherein the labeled nucleic acid comprises a contiguous nucleotide sequences which is completely complementary to a part of the nucleotide sequence of said neuregulin 1-associated gene 1 nucleic acid.

56. A reagent kit for assaying a sample for the presence of a neuregulin 1-associated gene 1 nucleic acid, comprising one or more nucleic acids comprising a contiguous nucleotide sequence which is at least partially complementary to a part of the nucleotide sequence of said neuregulin 1-associated gene 1 nucleic acid, and which is capable of acting as a primer for said neuregulin 1-associated gene 1 nucleic acid when maintained under conditions for primer extension.

57. The use of a nucleic acid which is 100 or fewer nucleotides in length and which is either: a) at least 80% identical to a contiguous sequence of nucleotides in SEQ ID NO: 1; b) at least 80% identical to the complement of a contiguous sequence of nucleotides in SEQ ID NO: 1; or c) capable of selectively hybridizing to said neuregulin 1-associated gene 1 nucleic acid, for assaying a sample for the presence of a neuregulin 1-associated gene 1 nucleic acid.

58. The use of a nucleic acid which is 100 or fewer nucleotides in length and which is either: a) at least 80% identical to a contiguous sequence of nucleotides in SEQ ID NO: 1; b) at least 80% identical to the complement of a contiguous sequence of nucleotides in SEQ ID NO: 1; or c) capable of selectively hybridizing to said neuregulin 1-associated gene 1 nucleic acid, for assaying a sample for the presence of a neuregulin 1-associated gene 1 nucleic acid that has at least one nucleotide difference from SEQ ID NO: 1.

59. The use of a nucleic acid which is 100 or fewer nucleotides in length and which is either: a) at least 80% identical to a contiguous sequence of nucleotides in SEQ ID NO: 1; b) at least 80% identical to the complement of a contiguous sequence of nucleotides in SEQ ID NO: 1; or c) capable of selectively hybridizing to said neuregulin 1-associated gene 1 nucleic acid, for diagnosing a susceptibility to schizophrenia.
